SEQUENCE LISTING (10 sequences)

SEQ ID NO: 1 - protein, 13 aa, Simian virus 40
CGGGPKKKRKVGG

SEQ ID NO: 2 - protein, 13 aa, Human adenovirus type 1
CGGFSTSLRARKA

SEQ ID NO: 3 - protein, 17 aa, Human adenovirus type 1
CKKKKKKSEDEYPYVPN

SEQ ID NO: 4 - protein, 27 aa, Human adenovirus type 1
CKKKKKKSEDEYPYVPNFSTSLRARKA

SEQ ID NO: 5 - protein, 10 aa, Human immunodeficiency virus
GRKKRRQRRR

SEQ ID NO: 6 - protein, 15 aa, Artificial Sequence
  Homo sapien Integrin binding domain with oligolysine residues
CKKKKKKGGRGDMFG

SEQ ID NO: 7 - protein, 5 aa, Artificial Sequence
  Synthetic RGD peptide
GRDSP

SEQ ID NO: 8 - protein, 8 aa, Homo sapiens
CRKRLDRN

SEQ ID NO: 9 - protein, 13 aa, Homo sapiens
SPRGDLAVLGHKY

SEQ ID NO: 10 - protein, 12 aa, Homo sapiens
TSPLNIHNGQKL

(19) United States
(12) Patent Application Publication
(10) Pub. No.: US 2011/0077581 A1
(43) Pub. Date: Mar. 31, 2011

(54) TARGETED CELLULAR DELIVERY OF NANOPARTICLES

(75) Inventors: ADEGBOYEGA K. OYELERE, MARIETTA, GA (US); MOSTAFA A. EL-SAYED, ATLANTA, GA (US); ERIK C. DREADEN, ATLANTA, GA (US)

(73) Assignee: GEORGIA TECH RESEARCH CORPORATION, ATLANTA, GA (US)

(21) Appl. No.: 12/890,519

(22) Filed: Sep. 24, 2010

Related U.S. Application Data
(60) Provisional application No. 61/245,725, filed on Sep. 25, 2009.

(51) Int. Cl. A61K 9/16; C07D 233/80; C07C 215/28; A61K 31/138; A61K 31/4166; A61P 35/00; A61K 31/335; A61N 5/06

(52) U.S. Cl. 604/20; 548/316.7; 564/317; 514/651; 514/391; 424/490; 514/449

(57) ABSTRACT

The various embodiments of the present disclosure relate generally to compositions and methods for the targeted cellular delivery of nanoparticles. More particularly, the various embodiments of the present invention are directed to the cellular delivery of nanoparticles tethered to a ligand by way of a poly(ethylene glycol) linkage, wherein the ligand demonstrates a binding specificity for a cellular target. In an exemplary embodiment, the ligand is tamoxifen and the cellular target is the estrogen receptor, which is upregulated in many breast cancer cells.

### CROSS-REFERENCE TO RELATED APPLICATION

This application claims, under 35 U.S.C. §119(e), the benefit of U.S. Provisional Application Ser. No. 61/245,725, filed 25 Sep. 2009, the entire contents and substance of which are hereby incorporated by reference as if fully set forth below.

### STATEMENT REGARDING FEDERALLY SPONSORED RESEARCH

This invention was made with U.S. Government support under Centers of Cancer Nanotechnology Excellence Award U54CA119338 awarded by the National Cancer Institute, Grant No. DE-FG02-97 ER14799 awarded by the U.S. Department of Energy, and Grant No. CHE-0554668 awarded by the National Science Foundation. The U.S. Government has certain rights in the invention.

### BACKGROUND OF THE INVENTION

1. Technical Field

The various embodiments of the present disclosure relate generally to compositions and methods for the targeted cellular delivery of nanoparticles. More particularly, the various embodiments of the present invention are directed to cellular delivery of nanoparticles tethered to ligands having specificity for a cellular target.

2. Description of Related Art

Binding of the steroidal hormone 17β-estradiol (E₂) to estrogen receptor (ER) is a process essential to normal cell proliferation and differentiation in women. E₂ binding induces a conformational change in ER which allows it to recruit cofactors necessary for the transcription of various genes commonly upregulated in malignant cells (1) (e.g. transforming growth factor alpha (2), c-myc (3), and cathepsin D (4). Accordingly, hormone receptors such as ER or progesterone receptor are overexpressed in 75-80% of all breast cancers (5). Anti-estrogen compounds, such as the small molecule breast cancer treatment drug tamoxifen (TAM) compete with E₂ for binding to ER, conformationally preventing adoption of associated transcription cofactors and subsequently initiating programmed cell death (6-9).

Diagnostic and therapeutic applications of functionalized nanoparticles are highly attractive due to the inherently multivalent nature of their surface (10-14). Like divalent antibodies, the binding affinity of a nanoparticle conjugate is enhanced proportional to the density of its binding sites. Receptor-mediated therapeutic response (i.e. potency) is similarly increased as a function of local ligand concentration and in cases where intracellular drug transport relies on passive diffusion, uptake of nanoparticle conjugates can greatly increase delivery rates (15, 16). Enhanced permeability and retention (EPR) of nano-sized drug conjugates can also lead to augmented and preferential accumulation at tumor sites in vivo (17, 18). Due to their biocompatibility (19, 20), stability (21), and potential use in photothermal laser treatments (18, 22-26), gold nanoparticles are excellent candidates for such ligand-receptor targeting strategies of cancer treatment.

Selective targeting and delivery of gold nanoparticles functionalized with ligands of cell surface receptors overexpressed by malignant cells has been well documented. Huang et al. have shown that oral cancer cells upregulating human epidermal growth factor receptor (EGFR, HER1, ErbB1) can be selectively labeled and photothermally destroyed by gold nanospheres and nanorods targeted with IgG antibodies (24, 27). ScFv fragments of anti-EGFR have also been used to selectively target and accumulate gold nanoparticles at tumor sites in vivo (28). Folate receptor has been employed to selectively deliver gold nanospheres to malignant cells in vitro (29), while Wei and coworkers have similarly demonstrated selective uptake and photothermal therapy of cancer cells using gold nanorods functionalized with a thiol-polyethylene glycol folate derivative (25, 30).

While the enhanced cellular uptake and selective delivery achieved with many gold nanoparticles conjugates are promising, their tumor selectivity is derived largely from the EPR effect, occasioned by the leaky tumor vasculature. However, not all tumors are amenable to the EPR effect, especially in regard to the delivery of the nanoparticles of relatively large size. Where active targeting has been demonstrated, nanoparticles are often delivered to either the tumor cell surface, the cytosol, or trapped in an endosome. The subcellular localization of gold nanoparticles is very important for several of their biological activities. For example, the efficacy of gold nanoparticles in binary cancer therapy, such as photothermal therapy, is enhanced upon their accumulation in and around the nucleus. A major drawback of several of the current nuclear-membrane targeting gold nanoparticles conjugates is their inability to distinguish between the nucleus of normal cells and tumor cells.

In order to achieve the selective delivery of gold nanoparticles to the nucleus of cancer cells, novel platforms for the targeted cellular delivery of nanoparticles and therapeutics are needed. The focus of the current application is to such novel platforms for the targeted cellular delivery of nanoparticles and therapeutics to tumor cells overexpressing endocrine receptors, such as the estrogen receptor and the androgen receptor.

### BRIEF SUMMARY OF THE INVENTION

Various embodiments of the present invention are directed to compositions and methods for the targeted cellular delivery of nanoparticles. More specifically, the various embodiments of the present invention are directed to the cellular delivery of nanoparticles tethered to ligands having specificity for a cellular target, such as a tumor cell.

An aspect of the present invention includes a therapeutic platform for targeted cellular delivery comprising: a core; a ligand having specificity for a target; and a linker comprising a linking moiety, wherein the linker is attached to the ligand, and wherein the linking moiety attaches the linker to the core, effectively tethering the ligand to the core. In an exemplary embodiment, the core is a gold nanoparticle, the linker is a poly(ethylene glycol) derivative having a thiol functional group, the target is an endocrine receptor, and the ligand is an endocrine receptor antagonist. In one embodiment, the endocrine receptor is an estrogen receptor, and the ligand is tamoxifen. In another embodiment, the endocrine receptor is an androgen receptor, and the ligand is nilutimide. In some embodiments, the therapeutic platform can further include an active agent, a targeting moiety, or a combination thereof.

Another aspect of the present invention includes a method for delivering a therapeutic platform to a target cell. This method involves administering to a subject an effective amount of a therapeutic platform, the therapeutic platform comprising: a core; a ligand having specificity for a target; and a linker comprising a linking moiety, wherein the linker is attached to the ligand, and wherein the linking moiety attaches the linker to the core, effectively tethering the ligand to the core; and selectively targeting the therapeutic platform to a cell of the subject. In an exemplary embodiment, the core is a gold nanoparticle, the linker is a poly (ethylene glycol) derivative having a thiol functional group. In instances where the subject demonstrates a neoplastic pathology, the target can be an endocrine receptor, and the ligand can be an endocrine receptor antagonist. More specifically, in instances of breast cancer where a breast cancer cell is overexpressing an estrogen receptor, the endocrine receptor is an estrogen receptor, and the ligand is tamoxifen.

Embodiments of methods for delivering a therapeutic platform to a target cell can further include inducing selective cytotoxicity of a breast cancer cell overexpressing an estrogen receptor. Additionally, methods for delivering a therapeutic platform to a target cell described herein can further involve exposing the cell to light energy effective to generate heat from the gold nanoparticle; and thermally ablating the cell, such as a breast cancer cell or a prostate cancer cell.

Yet another aspect of the present invention includes a therapeutic platform for targeted cellular delivery comprising: a gold nanoparticle; a ligand having specificity for an endocrine receptor; a thiol-poly(ethylene glycol) linker, wherein the thiol-poly(ethylene glycol) linker is covalently attached to the gold nanoparticle via a thiol functional group, and wherein the ligand is conjugated to the thiol-poly(ethylene glycol) linker via an azide-alkyne coupling. In one embodiment, the ligand comprises tamoxifen, and the endocrine receptor comprises an estrogen receptor. In another embodiment, the therapeutic platform can further comprise taxol.

Other aspects and features of embodiments of the present invention will become apparent to those of ordinary skill in the art, upon reviewing the following description of specific, exemplary embodiments of the present invention in conjunction with the accompanying figures.

### BRIEF DESCRIPTION OF DRAWINGS

FIG. 1A is a schematic of the chemical synthesis of thiol-pegylated tamoxifen (TAM-PEG-SH).

FIG. 1B illustrates the covalent attachment of thiol-pegylated tamoxifen to 25 nm gold nanoparticles (AuNPs).

FIG. 1C is a schematic of the chemical synthesis of a first generation androgen receptor ligand poly(ethylene glycol)-thiol conjugate.

FIG. 1D is a schematic of the chemical synthesis of a second generation androgen receptor ligand poly(ethylene glycol)-thiol conjugate.

FIG. 1E provides a schematic of the conjugation of androgen receptor ligands to poly (ethylene glycol).

FIGS. 2A-B illustrate the structure of HNSCP-1 and HNSCP-2 and the functionalization of these peptides.

FIG. 3 depicts dark-field scattering microscopy showing ligand- and receptor-dependent intracellular targeting of breast cancer cells by gold nanoparticle conjugates. Representative dark-field scattering images of ERα(+) [MCF-7, top] and ERα(−) [MDA-MB-231, bottom] human adenocarcinoma cells incubated for 24 h with 1 μM TAM-PEG-SH AuNP (left) and PEG-SH AuNP (right) conjugates (ca. 1.2×10⁴ TAM-PEG-SH per AuNP).

FIG. 4A graphically depicts time-dependent dose-response curves for cell viability of estrogen receptor alpha positive [MCF-7] breast cancer cells incubated with equivalent concentrations of TAM-PEG-SH as a free drug. Error bars represent standard deviation.

FIG. 4B graphically depicts time-dependent dose-response curves for cell viability of estrogen receptor alpha positive [MCF-7] breast cancer cells incubated with equivalent concentrations of TAM-PEG-SH as a gold nanoparticle conjugate. Error bars represent standard deviation.

FIG. 4C demonstrates time-dependent IC₅₀ (50% inhibitory concentration) values, which show 1.3-2.7 times enhanced potency from the nanoparticle conjugate versus the free drug. 3.6, 1.4, 1.1 μM TAM-PEG-SH IC₅₀ (24, 36, 48 h, respectively) versus 6.4, 2.4, 1.3, 1.0, 0.88 μM TAM-PEG-SH AuNP IC₅₀ (6, 12, 24, 36, 48 h, respectively).

FIG. 5 depicts representative dark-field scattering and bright-field transmission image overlays of TAM-PEG-SH AuNP competitive binding following 24 h incubation with 17β-estradiol. ERα(+) breast cancer cells [MCF-7] were incubated overnight with increasing concentrations of estrogen, followed by 24 h incubation with 10 μM tamoxifen-gold nanoparticle conjugates.

FIG. 6 illustrates suppression of TAM-PEG-SH AuNP activity by estrogen competition in ERα(+) breast cancer cells. Growth inhibition of MCF-7 cells incubated for 24 h with 10 μM TAM-PEG-SH AuNPs when previously untreated (left) and treated overnight with 10 μM 17β-estradiol (right).

FIG. 7 graphically depicts in vitro laser photothermal therapy of estrogen receptor (+) human breast cancer cells.

### DETAILED DESCRIPTION OF THE INVENTION

The present invention may be understood more readily by reference to the following Detailed Description of the Invention and the Examples included therein. Before the present compositions and methods are disclosed and described, it is to be understood that this invention is not limited to any specific ligands, specific targets, specific cell types, specific disease states, specific active agents, etc., as such may, of course, vary, and the numerous modifications and variations therein will be apparent to those skilled in the art. Throughout this description, various components can be identified as having specific values or parameters, however, these items are provided as exemplary embodiments. Indeed, the exemplary embodiments do not limit the various aspects and concepts of the present invention as many comparable parameters, sizes, ranges, and/or values can be implemented. The terms “first,” “second,” and the like, “primary,” “secondary,” and the like, do not denote any order, quantity, or importance, but rather are used to distinguish one element from another. Further, the terms “a,” “an,” and “the” do not denote a limitation of quantity, but rather denote the presence of “at least one” of the referenced item. Thus, for example, reference to “a ligand” can mean that one or more than ligand can be utilized.

An aspect of the present invention includes a platform for targeted cellular delivery. The platform includes a core, a ligand having specificity for a target, and a linker that tethers the ligand to the core. As used herein, the term “core” is intended to encompass all amorphous and crystalographically ordered particulates regardless of their shape and having an average longest dimension of about 1 nanometer to about 5 micrometers. A core can be made of many materials, including, but not limited to, individual elements, (e.g., metals, metalloids, and non-metals); binary compounds, multinary compounds, alloys, polymers, composite or hybrid materials, any of which can demonstrate metallic, semiconducting, or dielectric/insulting behavior.

In a preferred embodiment, a core comprises a nanoparticle. As used herein the term “nanoparticle” is intended to encompass all amorphous and crystalographically ordered particulates regardless of their shape and having an average longest dimension of less than or equal to about 1000 nm. This includes individual element particulates, (e.g., metals, metalloids, and non-metals); binary compound particulates, multinary compound particulates, alloy particulates, polymeric particulates, composite or hybrid particulates, and the like. The term nanoparticles is also intended to encompass a variety of shapes, including, but not limited to solid spheres, hollow spheres, spherical core-shells, solid rods, hollow rods, solid cubes, solid cubic cages, solid stars, solid triangular prismatic plates (e.g., nanopyramids), solid ellipsoids, hollow ellipsoids, core-shell ellipsoids, solid rings, solid hemispheres, solid circular disks, solid ellipsoidal rings, among others.

In a particularly preferred embodiment, the nanoparticle comprises a metal. The metal may be selected from a metal in groups IA, IB, IIB and IIIB of the periodic table, as well as the transition metals, especially those of group VIII. Preferred metals include gold, silver, aluminum, platinum, copper, ruthenium, zinc, iron, nickel, and calcium. Other suitable metals also include the following in all of their various oxidation states: lithium, sodium, magnesium, potassium, scandium, titanium, vanadium, chromium, manganese, cobalt, gallium, strontium, niobium, molybdenum, palladium, indium, tin, tungsten, rhenium, and gadolinium. The metals are preferably provided in ionic form and derived from an appropriate metal compound.

A preferred metal is gold. In one embodiment, the gold nanoparticles have a negative charge at an approximately neutral pH. It is thought that this negative charge prevents the attraction and attachment of other negatively charged molecules. In contrast, positively charged molecules are attracted to and bind to the gold particle. In such preferred embodiment, a gold nanoparticle may have an average longest dimension of about 1 nanometer to about 1,000 nanometers, and more preferably about 5 nanometers to about 100 nanometers. In an exemplary embodiment, a gold nanoparticle comprises a solid sphere having an average hydrodynamic diameter of about 25 nanometers to about 50 nanometers.

The core, which preferably comprises a nanoparticle, is tethered to a ligand. As used herein, the term “ligand” refers to a biomolecule or a chemical entity having a capacity or affinity for binding to a target. A ligand can include many organic molecules that can be produced by a living organism or synthesized, for example, a protein or portion thereof, a peptide, a polysaccharide, an oligosaccharide, a sugar, a glycoprotein, a lipid, a phospholipid, a polynucleotide or portion thereof, an oligonucleotide, an aptamer, a nucleotide, a nucleoside, DNA, RNA, a DNA/RNA chimera, an antibody or fragment thereof (e.g., Fab, scFv), a receptor or a fragment thereof, a receptor ligand, a nucleic acid-protein fusion, a hapten, a nucleic acid, a virus or a portion thereof, an enzyme, a co-factor, a cytokine, a chemokine, as well as small molecules (e.g., a chemical compound), for example, primary metabolites, secondary metabolites, and other biological or chemical molecules that are capable of activating, inhibiting, or modulating a biochemical pathway or process, and/or any other affinity agent, among others. A ligand can come from many sources, including libraries, such as small molecule libraries, phage display libraries, aptamer libraries, or any other library as would be apparent to one of ordinary skill in the art after review of the disclosure of the present invention. In an embodiment of the present invention, a platform for targeted cellular delivery can further comprise two or more types of ligands, which may be referred to as a multi-ligand platform or approach.

As used herein, a “target” or “target molecule” refers to a biomolecule that could be the focus of a therapeutic drug strategy, diagnostic assay, or a combination thereof, sometimes referred to as a theranostic. Therefore, a target can include, without limitation, many organic molecules that can be produced by a living organism or synthesized, for example, a protein or portion thereof, a peptide, a polysaccharide, an oligosaccharide, a sugar, a glycoprotein, a lipid, a phospholipid, a polynucleotide or portion thereof, an oligonucleotide, an aptamer, a nucleotide, a nucleoside, DNA, RNA, a DNA/RNA chimera, an antibody or fragment thereof, a receptor or a fragment thereof, a receptor ligand, a nucleic acid-protein fusion, a hapten, a nucleic acid, a virus or a portion thereof, an enzyme, a co-factor, a cytokine, a chemokine, as well as small molecules (e.g., a chemical compound), for example, primary metabolites, secondary metabolites, and other biological or chemical molecules that are capable of activating, inhibiting, or modulating a biochemical pathway or process, and/or any other affinity agent, among others.

The ligand may be, for example, one member of a biointeractive complex that comprises two or more biomolecules that have a binding affinity for one another. Consequently, the target may also be one member of such a biointeractive complex that demonstrates binding affinity for the ligand. Examples of biointeractive complexes (e.g., ligand-target complexes) can include for example, a protein:protein complex, a protein:peptide complex, a polynucleotide:polynucleotide complex, a polynucleotide:oligonucleotide complex, an oligonucleotide:protein complex, a polynucleotide:protein complex; a small molecule:receptor complex, a peptide:polynucleotide complex, a peptide: oligonucleotide complex; an antibody:antigen complex, an enzyme:substrate complex, or a biomolecule:drug complex, among others.

The phrase “having specificity for a target” with respect to the ligand as used herein can also be referred to as the “binding activity” or “binding affinity” of the ligand relative to the target. These phrases may be used interchangeably herein and are meant to refer to the tendency of a ligand to bind or not to bind to a target. The energetics of these interactions are significant in “binding activity” and “binding affinity” because they define the necessary concentrations of interacting ligands and targets, the rates at which these ligands and targets are capable of associating, and the relative concentrations of bound and free ligands and targets. The energetics are characterized through, among other ways, the determination of a dissociation constant, K_{d}. The specificity of the binding is defined in terms of the comparative dissociation constants (K_{d}) of the ligand for target as compared to the dissociation constant with respect to the ligand and other materials in the cellular environment or unrelated molecules in general. Typically, the K_{d} for the ligand with respect to the target will be 2-fold, preferably 5-fold, more preferably 10-fold less than K_{d} with respect to target and the unrelated material or accompanying material in the cellular environment. Even more preferably, the K_{d} will be 50-fold less, more preferably 100-fold less, and more preferably 200-fold less than K_{d} with respect to target and the unrelated material or accompanying material in the cellular environment.

In an exemplary embodiment of the present invention, the target comprises a cellular receptor and the ligand comprises a biomolecule or a chemical compound that has specificity for the receptor. In a preferred embodiment, the target is a cellular receptor that is specifically overexpressed in cells exhibiting a neoplastic disease. Neoplastic disease may occur in any organ or tissue including, but not limited to, bone, brain, breast, cervix, colon, endometrium, esophagus, eye, gallbladder, head and neck, kidney, liver, lung, lymphoid, mucosal, neuronal, ovary, pancreas, prostate, rectal, skin, stomach, and/or testicles, among others. The term “neoplastic disease” is intended to refer to cells that have uncontrolled growth, hyperplasia, tumors, tumorigenesis, cancer, metastasis, and the like. A person of ordinary skill in the art would readily realize that neoplastic disease presetning different tissue or organ tropisms may overexpress different cellular receptors. For example, overexpression of the estrogen receptor is associated with breast cancer, whereas overexpression of the androgen receptor is associated with prostate cancer. Thus, a person of ordinary skill in the art would realize that different ligands may be used to target different types of neoplastic disease.

In an exemplary embodiment, the target comprises an estrogen receptor and the ligand comprises a selective estrogen receptor modulator (SERM), an anti-estrogen, an aromatase inhibitor, or a combination thereof. An SERM or anti-estrogen compound can include, but is not limited to, tamoxifen, triphenylethylene, raloxifene, arzoxifene, basedoxifene, lasofoxifene, toremifene, triparanol, ethamoxytriphetol, trianisylchlorethylene, clomiphene, nafoxidine, fulvestrant, anastrozole, letrozole, exemestane, and derivatives thereof. In a preferred exemplary embodiment, the ligand comprises tamoxifen. In another exemplary embodiment, the ligand can comprise a tamoxifen metabolite, such as N-desmethyltamoxifen, N-desdimethyltamoxifen, metabolite Y, endoxifene, or 4-hydroxytamoxifen. In another exemplary embodiment, the target comprises an androgen receptor and the ligand comprises an anti-androgen. Examples of anti-androgens include nilutamide, bicalutamide, filutamide, MDV3100, RD1631, and derivatives thereof.

As discussed above, the ligand is tethered to the core by way of a linker. Generally, the linker comprises a compound that is capable of one or more of: facilitating covalent, non-covalent, or electrostatic attachment to the surface of the core (e.g., nanoparticle); increasing the biocompatibility of the platform; increasing aqueous stability of the platform; minimizing non-specific cell binding; and preventing opsonization of the platform and subsequent clearance by the reticulo-endothelial system. In one embodiment of the present invention, the linker comprises poly(ethylene glycol) (PEG) and more preferably a PEG derivative. The PEG linker or PEG derivative linker can have many topologies including, but not limited to, a branched topology, a graft topology, a comb topology, a star topology, a cyclic topology, a network topology, or combinations thereof, among others.

According to the embodiments of the invention, PEG or a PEG derivative comprises repeat unit ranging from about 1 to about 5,000, and preferably from about 4 to about 100, and more preferably from about 5 to about 20. A “PEG derivative” refers to a poly(ethylene glycol) molecule that has been altered by the addition of a functional group, a chemical entity, or the like, which facilitates attachment to the core. Examples of suitable functional groups include a thiol group, a vicinal dithiol group, thiocyanate group, an amine group, a carboxyl group, selenium, iodine, and silicate, among others. A person of ordinary skill in the art would realize that the type of PEG derivative employed in the platform depends upon the chemical composition of the core. For example, thiol-PEG works well with cores comprising a metal or a semiconductor, whereas carboxyl-PEG work well for cores comprising oxides. In a preferred embodiment, where the core comprises a gold nanoparticle, the preferred linker is a thiol-derivatized PEG having a repeat unit of about 4 to about 100.

Although not wishing to be bound to any particular theory, it is thought that the PEG linker provides aqueous stability to the platform for targeted cellular delivery, as many of the ligands demonstrate some hydrophobic properties. In addition, it is thought that the PEG tether provides a degree of flexibility to the ligand, promoting the association of one or more ligands with one or more target cellular receptors, effectively inducing receptor clustering and subsequent receptor-mediated endocytosis.

Irrespective of the type of cores, in some embodiments of the present invention, all of the attachment sites of the core are bound to a linker. In such embodiments, the ratio of linker to attachment sites on the core is equal to about 1. In other embodiments of the present invention, only a portion of the attachment sites of the core are bound to a linker or a linker may be bound to multiple attachment sites (e.g., a branched PEG). Consequently, the ratio of linker to attachment sites may be less than about 1. Further, on a given core, one or more types of linkers may be bound to the core.

In another embodiment of the present invention, the linker can be configured to release the ligand upon exposure to a stimulus, such as for example, enzymes, proteases, light, heat, magnetic fields, changes in pH, and RF radiation, among others. For example, stimuli can include proteases, such as Factor X, esterases, matrix metalloproteases (e.g., MMP-2 and MMP-9), among others.

In addition to being bound to the core, the linker is also conjugated to the ligand. In many embodiments of the present invention, the linker is conjugated directly to the ligand; however, embodiments of the present invention also contemplate indirect conjugation of the ligand to the linker. A person of ordinary skill in the art would realize that conjugation of a ligand to a linker depends on the chemical composition of the ligand and the linker.

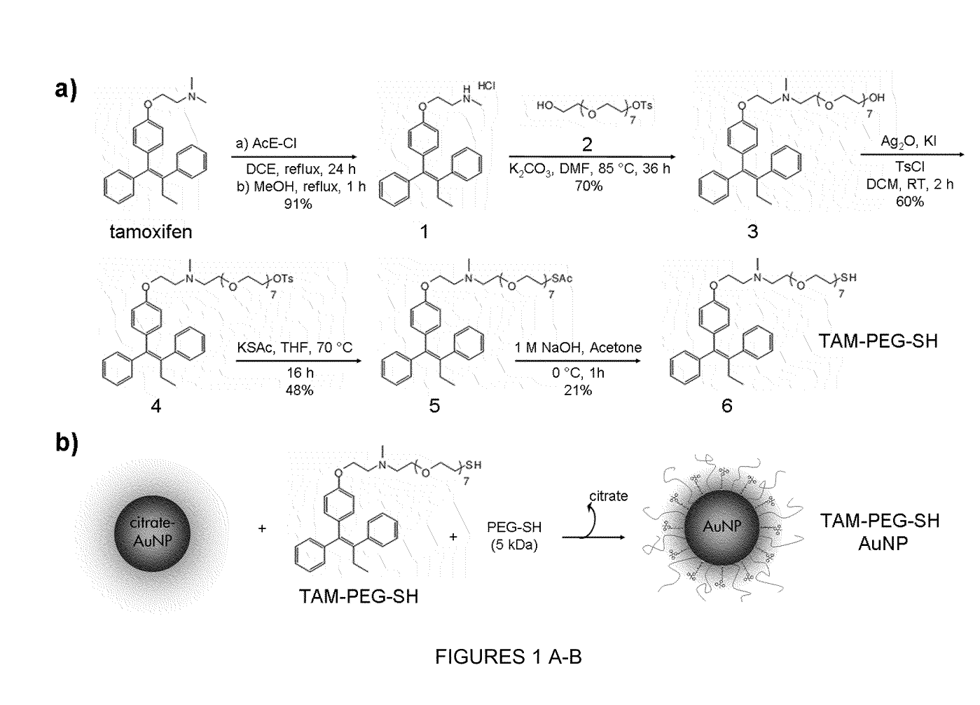

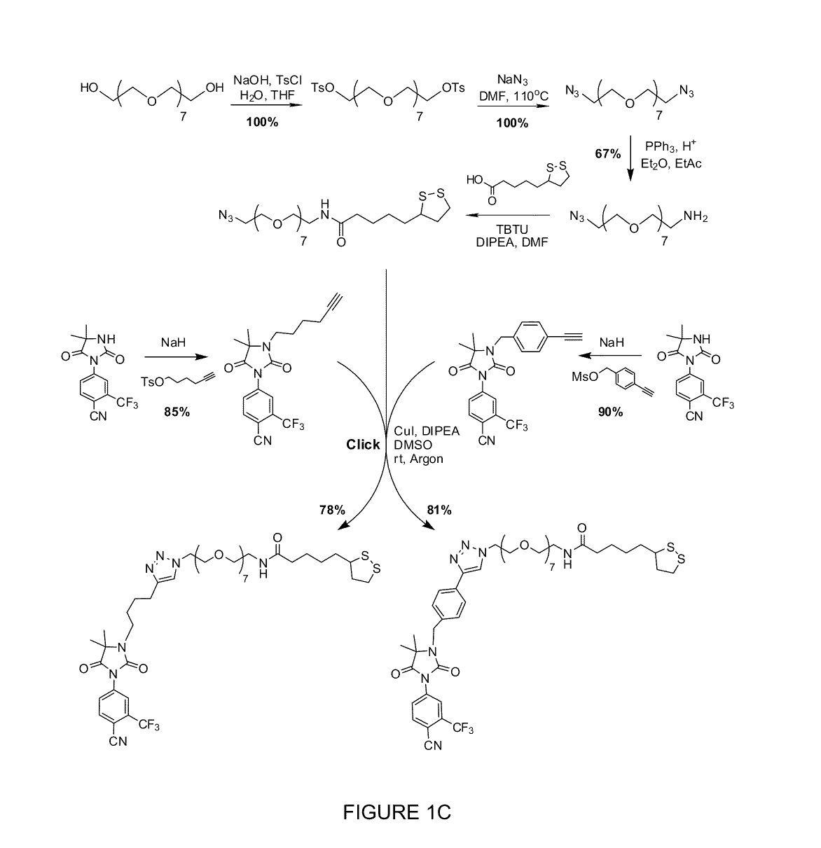

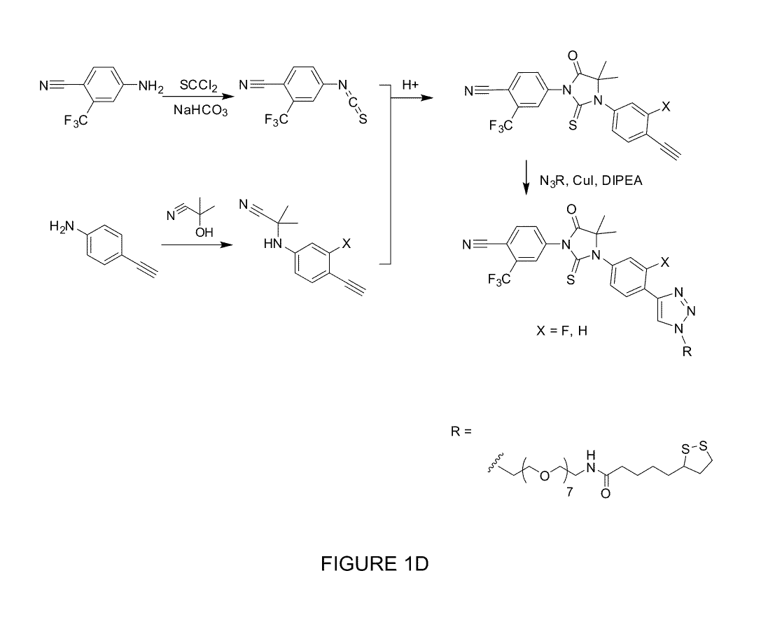

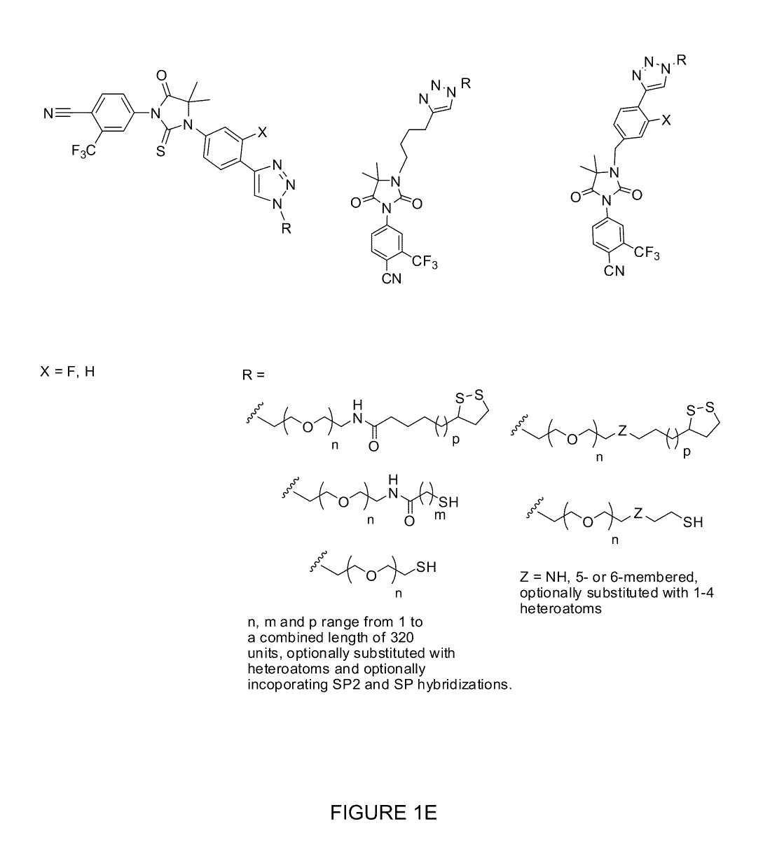

For example, in the case of ligand, tamoxifen, PEG was conjugated to tamoxifen through a five-step synthetic route (FIG. 1A). This synthetic process comprised the N-demethylation of tamoxifen, which yielded N-desmethyltamoxifen. Alkylation of N-desmethyltamoxifen with mono-tosylated octaethylene glycol provided pegylated tamoxifen. Subsequent tosylation of pegylated tamoxifen followed by thioacetylation and base treatment of the intermediate product resulted in the synthesis of tamoxifen conjugated to the thiol-PEG derivative.

In the case of a first generation androgen receptor ligand, the ligand can be conjugated to PEG through a synthetic process shown in FIG. 1C. In the case of a second generation androgen receptor ligand, the ligand can be conjugated to PEG through a synthetic process shown in FIG. 1D, which is further discussed in Jung, M. E.; Ouk, S.; Yoo, D.; Sawyers, C. L.; Chen, C.; Tran, C.; Wongvipat, J., Structure-Activity Relationship for Thiohydantoin Androgen Receptor Antagonists for Castration-Resistant Prostate Cancer (CRPC). J. Med. Chem. 2010, 53 (7), 2779-2796, which is hereby incorporated by reference. Additional examples of conjugation of androgen receptor ligands to PEG can be found in FIG. 1E.

Thus, the present invention provides a method of manufacture for a tamoxifen-poly(ethylene glycol)-thiol conjugate, a method of manufacture for a first generation androgen receptor ligand poly(ethylene glycol)-thiol conjugate, and a method of manufacture for a second generation androgen receptor ligand poly(ethylene glycol)-thiol conjugate.

In another embodiment, a platform for targeted cellular delivery can further comprise an active agent. As used herein, the term “active agent” can include, without limitation, agents for gene therapy; analgesics; anti-arthritics; anti-asthmatic agents; anti-cancer agents; anti-cholinergics; anti-convulsants; anti-depressants; anti-diabetic agents; anesthetics; antibiotics; antigens; anti-histamines; anti-infectives; anti-inflammatory agents; anti-microbial agents; anti-fungal agents; anti-nauseants; anti-neoplastics; anti-Parkinson agents; anti-spasmodics; anti-pruritics; anti-psychotics; anti-pyretics; anti-viral agents; nucleic acids; DNA; RNA; siRNA; polynucleotides; nucleosides; nucleotides; amino acids; peptides; proteins; carbohydrates; lectins; lipids; fats; fatty acids; viruses; immunogens; antibodies and fragments thereof; sera; immunostimulants; immunosuppressants; cardiovascular agents; channel blockers (e.g., potassium channel blockers, calcium channel blockers, beta-blockers, alpha-blockers); anti-arrhythmics; anti-hypertensives; inhibitors of DNA, RNA, or protein synthesis; neurotoxins; vasodilating agents; vasoconstricting agents; gases, growth factors; growth inhibitors; hormones; steroids; steroidal and non-steroidal anti-inflammatory agents; corticosteroids; angiogenic agents; anti-angiogenic agents; hypnotics; muscle relaxants; muscle contractants; sedatives; tranquilizers; ionized and non-ionized active agents; metals; small molecules; pharmaceuticals; hemotherapeutic agents; wound healing agents; indicators of change in the bio-environment; enzymes; enzyme inhibitors; nutrients; vitamins; minerals; coagulation factors; anticoagulants; anti-thrombotic agents; neurochemicals (e.g., neurotransmitters); cellular receptors; radioactive materials; contrast agents (e.g., fluorescence, magnetic, radioactive); vaccines; modulators of cell growth; modulators of cell adhesion; modulators of cellular signaling cascades; cell response modifiers; chemical or biological materials or compounds that induce a desired biological or pharmacological effect; and combinations thereof.

In an exemplary embodiment, a platform for targeted cellular delivery comprises an anti-cancer active agent, including, but not limited to, Tykerb® (Lapatinib Ditosylate), Herceptin® (Trastuzumab), Taxol® (Paclitaxel), Purinethol® (6-mercaptopurine), Gemzar® (Gemcitabine), Photofrin® (Porfimer), methotrexate, among other anti-cancer or cytotoxic pharmaceuticals, many of which are described by the National Cancer Institute (NCl) website (http://www.cancer.gov/cancertopics/drug info/alphalist), which is hereby incorporated by reference in its entirety.

In another exemplary embodiment, the active agent comprises a reporter molecule, which includes many diagnostic or imaging agents. As used herein, a “reporter molecule” is a detectable compound or composition that is conjugated directly or indirectly to another molecule (such as the ligand or core) to facilitate detection of the platform. Specific, non-limiting examples of reporter molecules include fluorescent and fluorogenic moieties, enzymatic moieties, haptens, metallic, semiconducting, or dielectric particles (e.g., gold, iodine, gadolinium, or iron oxide), affinity tags, radioactive isotopes, and radiopharmaceuticals, among others. The reporter molecule can be directly detectable (e.g., optically detectable) or indirectly detectable (for example, via interaction with one or more additional molecules that are in turn detectable).

In one embodiment, the active agent can be coupled to the platform for targeted cellular delivery by being directly or indirectly bound to the core. For example, in embodiments where the core comprises a nanoparticle, conjugation of the active agent to the nanoparticle can utilize similar functional groups that are employed to tether PEG to the nanoparticle. Thus, the active agent can be directly bound to the nanoparticle through functionalization of the active agent. Alternatively, the active agent can be indirectly bound to the nanoparticle through conjugation of the active agent to a functionalized PEG, as discussed above. In another embodiment, the active agent can be structurally independent of the platform and can be co-administered with the platform.

In yet another embodiment, a platform for targeted cellular delivery can further comprise a targeting moiety. As used herein, the term “targeting moiety” refers to a substance associated with the core that enhances binding, transport, accumulation, residence time, bioavailability, or modifies biological activity or therapeutic effect of the platform, or its associated ligand and/or active agent in a cell or in the body of a subject. A targeting moiety can have functionality at the tissue, cellular, and/or subcellular level.

The targeting moiety can include, but is not limited to, an organic or inorganic molecule, a peptide, a peptide mimetic, a protein, an antibody or fragment thereof, a growth factor, an enzyme, a lectin, an antigen or immunogen, viruses or component thereof, a viral vector, a receptors, a receptor ligand, a toxins, a polynucleotide, an oligonucleotide or aptamer, a nucleotide, a carbohydrate, a sugar, a lipid, a glycolipid, a nucleoprotein, a glycoprotein, a lipoprotein, a steroid, a hormone, a growth factor, a chemoattractant, a cytokine, a chemokine, a drug, or a small molecule, among others.

In an exemplary embodiment of the present invention, the targeting moiety enhances binding, transport, accumulation, residence time, bioavailability, or modifies biological activity of the modifies biological activity or therapeutic effect of the platform, or its associated ligand and/or active agent in a neoplastic cell or in the body of a subject having a neoplastic disease. Thus, the targeting moiety can have specificity for cellular receptors associated with neoplastic disease. Consequently, a ligand, as described above, can be both a ligand and a targeting moiety.

The targeting moiety can be coupled to the platform for targeted cellular delivery by being directly or indirectly bound to the core. For example, in embodiments where the core comprises a nanoparticle, conjugation of the targeting moiety to the nanoparticle can utilize similar functional groups that are employed to tether PEG to the nanoparticle. Thus, the targeting moiety can be directly bound to the nanoparticle through functionalization of the targeting moiety. Alternatively, the targeting moiety can be indirectly bound to the nanoparticle through conjugation of the targeting moiety to a functionalized PEG, as discussed above. A targeting moiety can be attached to core by way of covalent, non-covalent, or electrostatic interactions.

For example, the platforms of the present invention may utilize peptide targeting sequences such as SV40 large T antigen nuclear localization signal (NLS), CGGGPKKKRKVGG (SEQ ID NO 1); the adenoviral NLS peptide, CGGFSTSLRARKA (SEQ ID NO 2); the adenoviral receptor-mediated endocytosis peptide, CKKKKKKSEDEYPYVPN (SEQ ID NO 3); the adenoviral fiber protein, CKKKKKKSEDEYPYVPNFSTSLRARKA (SEQ ID NO 4); the HIV-1 Tat NLS peptide, GRKKRRQRRR (SEQ ID NO 5); the integrin binding domain (RGD) with oligolysine residues, CKKKKKKGGRGDMFG (SEQ ID NO 6); a synthetic RGD peptide or polypeptide, GRDSP (SEQ ID NO 7); and an AP peptide, CRKRLDRN (SEQ ID NO 8). Additional exemplary targeting moieties include interleukin receptor, mucin-1, platelet derived growth factor receptor, fibroblast growth factor receptor, vascular endothelial growth factor receptor, lysophosphatidic acid receptor, and endothelin A peptide receptor, among others.

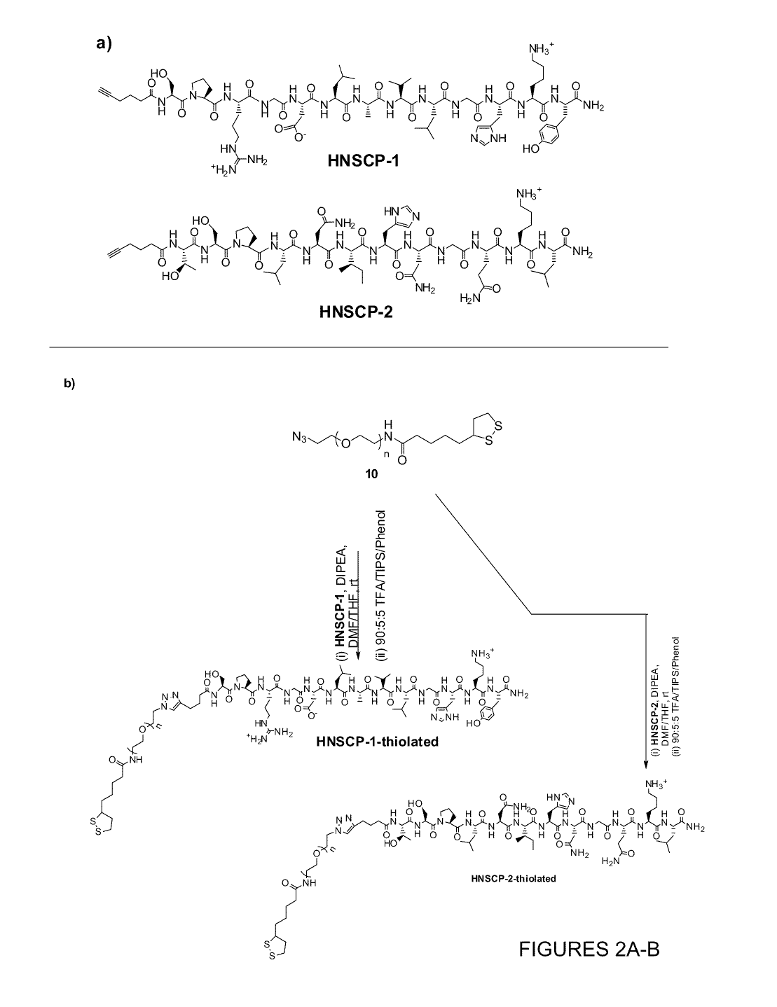

A person of ordinary skill in the art would realize that many targeting peptides could be employed in various embodiments of the present invention. For example, a targeting moiety can comprise a cancer targeting peptide, such as those disclosed by Aina, O. H., Sroka, T. C., Chen, M. L., Lam. K. S. Therapeutic cancer targeting peptides. Biopolymers 2002, 66, 184-99, which is hereby incorporated by reference. Examples of such cancer targeting peptides can be found in Hong, F. D., Clayman, G. L. Isolation of a Peptide for Targeted Drug Delivery into Human Head and Neck Solid Tumors. Cancer Res. 2000, 60, 6551-6556; and Nothelfer, E-M., Zitzmann-Kolbe, S., Garcia-Boy, R., Krämer, S., Herold-Mende, C., Altmann, A., Eisenhut, M., Mier, W., Haberkorn, U. Identification and Characterization of a Peptide with Affinity to Head and Neck Cancer. The Journal of Nuclear Medicine 2009, 50, 426-434, which are hereby incorporated by reference. For example, a peptide cancer targeting sequences can include the head and neck squamous cell cancer peptide-1 (HNSCP-1), SPRGDLAVLGHKY (SEQ ID NO 9); or HNSCP-2, TSPLNIHNGQKL (SEQ ID NO 10). FIG. 2A illustrates the structure of HNSCP-1 and HNSCP-2, and FIG. 2B illustrates the functionalization of the peptide.

Another aspect of the present invention comprises a method for delivering a platform for targeted cellular delivery to a target cell comprising: administering to a subject an effective amount of a therapeutic platform. As used herein, the term “subject” refers to animals and plants, as well as cells and tissues derived therefrom. For example, the systems and methods of the present invention are applicable to a broad range of animals, including, but not limited to, mammals, birds, fish, reptiles, amphibians, insects, preferably mammals, and more preferably humans. Mammals include, but are not limited to, humans, primates, horses, cows, sheep, pigs, goats, dogs, cats, rabbits, guinea pigs, rats, and mice.

Embodiments of the methods for delivering a platform for targeted cellular delivery of the present invention comprise administering an effective amount of platform. Administration of the platform may be performed by many known routes of administration, including, but not limited to, topical administration, oral administration, enteral administration, intratumoral administration, or parenteral administration (e.g., epifascial, intraarterial, intracapsular, intracardiac, intracutaneous, intradermal, intramuscular, intraorbital, intraosseous, intraperitoneal, intraspinal, intrasternal, intravascular, intravenous, intravesical, parenchymatous, or subcutaneous administration), among others.

In the methods for delivering a platform of the present invention and in the treatment of neoplastic disease, a therapeutically effective amount of a platform or a platform and active agent can be administered to a subject exhibiting a neoplastic disease. A “therapeutically effective amount” or “an effective amount” in the context of the present invention is considered to be any quantity of the platform or platform and active agent, which, when administered to a subject, causes prevention, reduction, remission, regression, or elimination of a neoplastic-related pathology, such as cell proliferation, tumorigenesis, and/or metastasis.

The amount of the platform or platform and active agent that can be used in the compositions or methods of the present invention can be determined using in vitro assays, as discussed in the below Examples, and by other methods known to those skilled in the art, such as pre-clinical and clinical trials. Furthermore, tolerable, therapeutically effective amounts of active agents, such as anti-cancer or cytotoxic pharmaceuticals, are known and can be obtained from the appropriate supplier or, for example, the U.S. Food and Drug Association (www.fda.gov).

An effective dose of a platform may be administered daily, more than one time a day, weekly, monthly, or over one or more years to treat neoplastic disease. An effective dose may comprise from about 0.001 μg to about 1,000 mg/kg subject of a platform, and more preferably from about 0.02 μg to about 200 mg/kg subject of a platform. Depending on the route of administration, the ligand, the specificity of the ligand for the target, and the presence or absence of an active agent, a preferable dosage would be one that would yield an adequate blood level or tissue fluid level in the subject that would effectively cause prevention, reduction, remission, regression, or elimination of a neoplastic-related pathology. For example, in the case of a platform comprising a tamoxifen-poly(ethylene glycol)-SH gold nanoparticle, an effective dose may comprise about 1.0 mg/day to about 40 mg/day, and preferably about 1.0 mg/day to about 20 mg/day, and more preferably about 1.0 mg/day to about 10 mg/day, and more preferably about 3.0 mg/day to about 5 mg/day.

The methods and compositions of the present invention may be used in combination with other treatments for neoplastic disease known in the art including, but not limited to, surgery, radiation therapy, chemotherapy, immunotherapy, photodynamic therapy, hyperthermia, and targeted therapies. In addition, the methods and compositions of the present invention can be utilized for treating a radiation-induced cell growth as well as for the treatment of cancers that demonstrate some resistance to a chemotherapeutic agent.

The methods and compositions of the present invention can further comprise photothermal therapy. Photothermal therapy involves the exposure of cells or tumors treated with the metallic nanoparticles platform to light energy (via laser irradiation), which is rapidly converted into heat energy on a picosecond time scale due to rapid electron-phonon and phonon-phonon processes. This heat energy generates a temperature increase that is sufficient to cause thermal destruction at the cellular and tissue level. Representative lasers include, but are not limited to CW argon lasers, CW Ti:Sapphire, dye, diode, as well as pulsed lasers. Considering that the platforms have ligands that demonstrate specificity for receptors selectively overexpressed in tumor cells, the nanoparticles platform provides a selective mechanism to thermally ablate tumor cells.

The methods and compositions of the present invention exhibit about can be implemented in treatment strategies for ER+/PR−/HER2− breast cancers, which are malignancies that historically exhibit poor tamoxifen response and poor HER2 expression/targeted treatment (e.g. Herceptin) response, and can make use of enhanced endocrine treatment potency. The methods and compositions of the present invention also contemplate multimodal breast cancer treatment strategies, which combine the selective cytostatic effects of the tamoxifen targeting moiety with selective administration of adjunctive: photothermal therapy (laser-assisted plasmonic photothermal therapy, PPTT), chemotherapy via co-functionalization, or small molecule kinase inhibition (e.g. dual tyrosine kinase inhibitors, Lapatinib/Tykerb) by co-functionalization, among others. The methods and composition of the present invention demonstrate: enhanced potency; selective delivery of co-functionalized therapeutic molecules; and selective delivery of laser-assisted plasmonic photothermal therapy (PPTT)

It should be understood, of course, that the foregoing relates only to exemplary embodiments of the present invention and that numerous modifications or alterations may be made therein without departing from the spirit and the scope of the invention as set forth in this disclosure. Therefore, while embodiments of this invention have been described in detail with particular reference to exemplary embodiments, those skilled in the art will understand that variations and modifications can be effected within the scope of the invention as defined in the appended claims. Accordingly, the scope of the various embodiments of the present invention should not be limited to the above discussed embodiments, and should only be defined by the following claims and all equivalents.

It should be noted that all patents, patent applications, and references included herein are specifically incorporated by reference in their entireties.

The present invention is further illustrated by way of the examples contained herein, which are provided for clarity of understanding. The exemplary embodiments should not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention or the scope of the appended claims.

### EXAMPLES

### Example 1

### Tamoxifen-Peg-Thiol Gold Nanoparticle Conjugates: Enhanced Potency and Selective Delivery for Breast Cancer Treatment

Like several members of the hormone receptor family, estrogen receptor (ER) isoforms are located both intracellularly and on the cell membrane (31-33). Gold nanoparticle analogs of the commercial pharmaceutical tamoxifen could therefore act not only as, selective targeting agents, but also as increasingly potent endocrine treatments for malignancies which overexpress ER (e.g. breast cancer). To this aim, a thiol-polyethylene glycol (PEG-SH) tamoxifen derivative was synthesized for subsequent gold nanoparticle (AuNP) conjugation (Scheme 1). A biocompatible (18, 34) PEG-SH linker was employed (i) to enable covalent attachment to the AuNP surface (Au—S 126 kJ*mol⁻¹) (35, 36), (ii) to minimize opsonin binding and reticulo-endothelial system uptake (37), (iii) to suppress non-specific cell binding/uptake (38) and protein adsorption (18, 21), and (iv) to afford stability (21) over a wide range of temperature, ionic strength, and pH.

The results presented below demonstrate enhanced potency and selective intracellular delivery of tamoxifen-targeted gold nanoparticles to ER(+) breast cancer cells in vitro. Particle uptake was observed in both a receptor- and ligand-dependent fashion with up to 2.7 fold enhanced drug potency versus the free drug. Both delivery and therapeutic response were shown to be suppressed by estrogen competition. Optical microscopy/spectroscopy and cell viability indicate that augmented growth inhibition versus the free drug can be attributed to increased rates of intracellular TAM transport by cellular uptake of the nanoparticle conjugate. Receptor- and ligand-dependent nanoparticle delivery suggests that the plasma membrane localized estrogen receptor alpha may facilitate selective particle uptake and presents future opportunities for co-administration of laser photothermal therapy (18, 22-26).

Synthesis of thiol-pegylated tamoxifen (TAM-PEG-SH). Octaethylene glycol (OEG), Tamoxifen, and all chemicals used in the synthesis were purchased from Sigma Aldrich. Anhydrous solvents and other reagents were purchased and used without further purification. Analtech silica gel plates (60 F₂₅₄) were used for analytical TLC, and Analtech preparative TLC plates (UV 254, 2000 μm) were used for purification. UV light was used to examine the spots. 200-400 Mesh silica gel was used in column chromatography. NMR spectra were recorded on a Varian-Gemini 400 magnetic resonance spectrometer. ¹H and ¹³C NMR spectra were recorded in parts per million (ppm) relative to the peaks of CDCl₃, (7.24 and 77.0 ppm, respectively). Mass spectra were recorded at the Georgia Institute of Technology mass spectrometry facility in Atlanta, Ga.

Synthesis of N-Desmethyl tamoxifen (1). The synthetic procedure was adapted from Olofson et al. (39). Briefly, tamoxifen (0.53 g, 1.43 mmol) was dissolved in anhydrous CH₂Cl₂ (15 ml) at 0° C. followed by addition of α-chloroethyl chloroformate (0.17 ml, 1.49 mmol). After 15 min at 0° C., the reaction was refluxed for 24 h. The solvent was evaporated off to obtain a yellowish oil, to which methanol (10 ml) was added and refluxed for approximately 3 h. The solvent was evaporated off, and purification performed by gel filtration using CH₂Cl₂, then 10:1 CH₂Cl₂/CH₃OH to obtain 0.52 g (91%) of N-desmethyl tamoxifen 2. ¹H NMR (DMSO, 400 MHz) δ 0.89 (3H, t, J=7.2 Hz), 2.43 (2H, q, J=14.8, 7.6 Hz), 2.56 (3H, s), 3.12 (2H, t, J=4.0 Hz), 4.08 (2H, t, J=4.8 Hz), 6.57 (2H, d, J=8.8 Hz), 6.76 (2H, d, J=8.8 Hz), 7.03-7.31 (10H, m), 9.56 (1H, br); HRMS [FAB, mnba] (C₂₅H₂₇NO)⁺ calcd, 358.2171. found, 358.2198.

Synthesis of Tosyl octaethylene glycol (2). The synthetic procedure was adapted from Bouzide and Sauvé (40). Briefly, octaethylene glycol (0.50 g, 1.35 mmol) was dissolved in anhydrous CH₂Cl₂ (7 ml) at 0° C., followed by addition of freshly prepared Ag₂O (0.47 g, 2.02 mmol), KI (0.09 g, 0.50 mmol), and then TsCl (0.26 g, 1.35 mmol). The reaction mixture was left to stir at 0° C., under argon for 30 min, after which TLC deemed the reaction complete. Ag₂O was filtered off over a pad of celite cake washing with 12:1 CH₂Cl₂/CH₃OH. The filtrate was concentrated and purified on a silica column using 3:2, then gradually 1:4 CH₂Cl₂/acetone to yield the title compound as a colorless oil (0.55 g, 78%). ¹H NMR (CDCl₃, 400 MHz) δ 2.44 (3H, s), 2.81 (1H, br), 3.58-3.70 (30H, m), 4.15 (2H, t, J=4.8 Hz), 7.35 (2H, d, J=8.4 Hz), 7.80 (2H, d, J=8.4 Hz); HRMS [ESI] (C₂₃H₄₀O₁₁S+H)⁺ calcd, 525.2364. found, 525.2377.

Synthesis of Tamoxifen-OEG-OH (3). N-Desmethyl tamoxifen (1) (0.27 g, 0.68 mmol) and tosyl octaethylene glycol (2) (0.54 g, 1.05 mmol) were dissolved in anhydrous DMF (10 ml), followed by addition of K₂CO₃ (0.95 g, 6.85 mmol), and stirred under argon at ˜85° C. for 24 h. DMF was evaporated off. Ethyl acetate was added to the residue and the resulting suspension was filtered off to remove excess K₂CO₃. Solvent was evaporated from the filtrate and the crude was purified by preparatory TLC using 12:1:0.1 CH₂Cl₂/CH₃OH/NH₄OH to obtain 0.342 g (70%) of compound 3 as an oil. ¹H NMR (CDCl₃, 400 MHz) δ 0.90 (3H, t, J=7.2 Hz), 1.85 (1H, br), 2.31 (3H, s), 2.44 (2H, q, J=14.0, 7.2 Hz), 2.64 (2H, t, J=6.0 Hz), 2.76 (2H, t, J=6.0 Hz), 3.53-3.72 (30H, m), 3.91 (2H, t, J=6.4 Hz), 6.54 (2H, m), 6.76 (2H, m), 7.10-7.40 (10H, m); ¹³C NMR (CDCl₃, 100 MHz) δ 13.8, 29.2, 43.3, 50.7, 56.6, 57.2, 61.7, 65.6, 69.1, 70.3, 70.5, 70.6, 70.7, 70.8, 73.0, 113.6, 126.2, 126.7, 128.0, 128.3, 129.7, 129.9, 132.1, 135.8, 138.4, 141.5, 142.6, 144.0, 156.8; HRMS [ESI] (C₄₁H₅₉NO₉+H)⁺ calcd, 710.4262. found, 710.4253.

Synthesis of Tamoxifen-OEG-Tosylate (4). Tamoxifen-OEG-OH (3) (0.33 g, 0.46 mmol) was dissolved in anhydrous CH₂Cl₂ (10 ml) at 0° C., followed by addition of Ag₂O (0.16 g, 0.69 mmol), KI (0.03 g, 0.18 mmol), and then TsCl (0.096 g, 0.5 mmol). Stirring was continued for 2 h at 0° C., then at room temperature, overnight. Ag₂O was filtered off through a pad of celite cake washing with ethyl acetate. Purification was performed on silica column eluting with 12:1 CH₂Cl₂/CH₃OH yielding the title compound as oil (0.24 g, 60%). ¹H NMR (CDCl₃, 400 MHz) δ 0.91 (3H, t, J=7.2 Hz), 2.31-2.46 (8H, m), 2.71 (2H, br), 2.83 (2H, br), 3.57-3.70 (28H, m), 3.95 (2H, br), 4.15 (2H, t, J=4.4 Hz), 6.53 (2H, d, J=8.4 Hz), 6.76 (2H, d, J=8.8 Hz), 7.10-7.34 (12H, m), 7.80 (2H, d, J=8.4 Hz); LRMS [ESI] (C₄₈H₆₅NO₁₁S+H)⁺ calcd, 864.1. found, 864.5.

Synthesis of Tamoxifen-OEG-SAc (5). KSAc (0.079 g, 0.69 mmol) was added to tamoxifen-OEG-Tosylate (4) (0.12 g, 0.14 mmol) dissolved in anhydrous THF and refluxed under argon at ˜75° C. for 16 h. TLC analysis indicated a substantial consumption of the starting material. THF was evaporated off, and the crude product dissolved in ethyl acetate. Decolorizing carbon was added and then filtered. Solvent was evaporated from the filtrate and the crude was purified by preparatory TLC using 12:1 CH₂Cl₂/CH₃OH to obtain 50 mg (48%) of 5 as reddish oil. ¹H NMR (CDCl₃, 400 MHz) δ 1.03 (3H, t, J=7.2 Hz), 2.45 (3H, s), 2.47 (3H, s), 2.56 (2H, q, J=15.2, 7.6 Hz), 2.80 (2H, t, J=6.0 Hz), 2.91 (2H, t, J=5.6 Hz), 3.21 (2H, t, J=6.0 Hz), 3.70-3.80 (28H, m), 4.05 (2H, t, J=6.0 Hz), 6.66 (2H, d, J=8.0 Hz), 6.88 (2H, d, J=8.0 Hz), 7.22-7.46 (10H, m); HRMS [ESI] (C₄₃H₆₁NO₉S+H)⁺ calcd, 768.4139. found, 768.4118.

Synthesis of Thiol-pegylated tamoxifen (6). Tamoxifen-OEG-SAc (5) (0.05 g, 0.065 mmol) was dissolved in acetone (1.5 ml) at 0° C., followed by addition of 1M NaOH (1.5 ml) and stirring continued at 0° C. for 7 h. The reaction mixture was quenched with water (20 ml), and then extracted with 20% CH₃OH in CH₂Cl₂ (4×15 mL). The organic layers were combined, dried under sodium sulfate, evaporated and purified on preparatory TLC using 11:1 CH₂Cl₂/CH₃OH to give 10 mg (21%) of the title compound as reddish semi-solid. ¹H NMR (CDCl₃, 400 MHz) δ 0.90 (3H, t, J=7.2 Hz), 2.32 (3H, s), 2.44 (2H, q, J=8.0, 7.6 Hz), 2.65 (2H, t, J=6.0 Hz), 2.76 (2H, t, J=6.4 Hz), 2.85 (2H, t, J=6.8 Hz), 3.53-3.72 (28H, m), 3.91 (2H, t, J=6.0 Hz), 6.52 (2H, d, J=8.8 Hz), 6.74 (2H, d, J=8.8 Hz), 7.08-7.32 (10H, m); ¹³C NMR (CDCl₃, 100 MHz) δ 13.5, 28.9, 29.6, 38.3, 43.3, 56.4, 57.0, 65.6, 69.2, 69.6, 70.3, 70.4, 70.5, 70.6, 113.3, 125.9, 126.4, 127.8, 128.0, 129.4, 129.6, 131.8, 135.4, 138.1, 141.2, 142.3, 143.7, 156.6; HRMS [ESI] (C₄₁H₅₉NO₈S]⁺ calcd, 725.3961. found, 725.4011.

Gold nanoparticle synthesis and TAM-PEG-SH conjugation. Gold nanoparticles (25 nm dia) were synthesized by Turkevich reduction of chloroauric acid (41). Briefly, 20 mL of 3.5 mg/mL aqueous sodium citrate was added to 200 mL of 1.0 mM aqueous HAuCl₄ under reflux, with stirring. The solution was refluxed for 15 min, then removed from the heat and stirred for an additional 30 min Excess sodium citrate was removed from the crude AuNP solution by centrifugation (13,000×g). TAM-PEG-SH (5 mg) was solubilized in 100 μL ethanol and diluted to 0.5 mM in deionized water. 0.5 mM PEG-SH (5 kDa, Lysan Bio) was solubilized in deionized water and PEG-SH or a 1:1 ratio TAM-PEG-SH and PEG-SH were added at a 1.4×10⁴-fold molar excess to a concentrated solution of citrate-capped AuNPs followed by overnight sonication. Particle concentration was estimated using the molar extinction coefficient for 23 nm citrate-capped gold nanospheres determined by Orendorff and Murphy (42) (1.3×10⁹ M⁻¹cm⁻¹). TAM-PEG-SH AuNP conjugates were dispersed in DMEM growth media supplanted with 10% v/v heat-inactivated fetal bovine serum, penicillin (100 U/ml), streptomycin (100 μg/ml), 4.5 g/L glucose, 4.5 g/L sodium pyruvate, without L-glutamine and phenol red to final ligand concentrations of 0.1, 0.5, 1, 2, 5, 10, and 20 μM and used immediately.

Gold Nanoparticle and Bioconjugate Characterization. Gold nanoparticles were analyzed by diffraction-contrast Transmission Electron Microscopy (TEM, JEOL 100CX II) and UV-Vis absorption spectroscopy (Ocean Optics, HR4000CG-UV-NIR). Absorption of TAM-PEG-SH at 280 nm was used to quantify the number of bound TAM-PEG-SH ligands per nanoparticle. An aqueous solution of gold nanospheres was incubated with a 1.4×10⁴-fold molar excess of both TAM-PEG-SH and PEG-SH overnight with sonication. Nanoparticle-conjugates were removed from solution by centrifugation (45 min, 13,000×g) and the observed change in UV absorption (280 nm) before and after nanoparticle conjugation was used to approximate the number of bound ligands. No contribution to absorption by PEG-SH was observed at these wavelengths and it assumed to occupy the majority of the remaining surface sites. Zeta potential of the gold nanoparticles and conjugates was measured using a NanoZS Zetasizer particle analyzer (Malvern) equipped with a 633 nm laser.

Cell culture and nanoparticle incubation. ERα(−) MDA-MB-231 and ERα(+) MCF-7 breast cancer cells (human adenocarcinoma, ATCC) or ERα(+) human squamous cell carcinoma (43-45) (HSC-3) cells were cultured to 10⁵ cells/cm² in DMEM growth media supplanted with 10% v/v heat-inactivated fetal bovine serum, penicillin (100 U/ml), streptomycin (100 μg/ml), 4.5 g/L glucose, 4.5 g/L sodium pyruvate, without L-glutamine and phenol red at 37° C. in a 5% CO₂ humidified atmosphere. Growth media was removed from the cell cultures and replaced with identical media containing gold nanoparticle conjugates heated to 37° C. at time=0 h.

Cell viability assay. Following incubation, growth media containing gold nanoparticle conjugates was removed and cells were rinsed twice in sterile Dulbecco's phosphate buffered saline (DPBS). Mitochondrial dehydrogenase activity was assessed by MTT or XTT spectrophotometric assay (Sigma TOX1, TOX2) following the manufacturer's instructions. The assay was performed using a SpectraMax Plus 384 microplate reader and statistical analysis was performed by t-test.

Selected-area absorption microspectrometry and dark-field scattering microscopy. Collagen-coated growth substrates were prepared by immersion of 18 mm dia glass coverslips in ethanol, followed by 30 min UV sterilization. Coverslips were immersed in a 0.22 μm filtered 0.04 mg/mL collagen (Roche) solution—prepared by solubilization in 5 mL 1% v/v aqueous acetic acid and dilution in 250 mL sterile DPBS for 6 h at 37° C. in a 5% CO₂ humidified atmosphere. The coated substrates were rinsed in sterile DPBS and placed in 12-well plates immediately prior to cell passage. Following incubation with gold nanoparticle conjugates, substrates were twice rinsed in sterile DPBS buffer and cells were fixed in cold 4% wt/wt paraformaldehyde in DPBS buffer for 15 min Coverslips were coated in glycerol, then mounted and sealed onto glass slides.

Dark-field microscopy was performed using an inverted objective Olympus 1×70 microscope fitted with a dark-field condenser (U-DCW), 100×/1.35 oil Iris objective (UPLANAPO), tungsten lamp, and a Nikon D200 digital SLR camera. Optical extinction spectra were obtained in a transmission configuration using a SEE110 absorption microspectrometer fitted with a pinhole aperture, fiber optic-coupled CCD array detector, 50× objective, and tungsten lamp. Periodic oscillations observed in some spectra are the result of interference between adjacent surfaces of the glass slides.

Results. The crude AuNP colloid (ca. 3 nM) was found by TEM to be predominantly comprised of 25 nm gold spheres exhibiting an extinction maximum at 530 nm. Based on the change in UV absorption (280 nm) of TAM solutions following nanoparticle conjugation and removal, we estimate 12,000 TAM-PEG-SH ligands per particle—41% of the maximum theoretical surface coverage for a 25 nm dia Au (111) surface. A change in zeta potential from −38.4 mV to −5.79 mV was also observed following TAM-PEG-SH functionalization. To preserve aqueous stability, TAM-PEG-SH AuNPs were not centrifuged prior to in vitro experiments, leaving 13% of free drug in solution. For comparison, concentrations for the nanoparticle conjugate are reported as effective ligand concentration (i.e. TAM-PEG-SH) throughout.

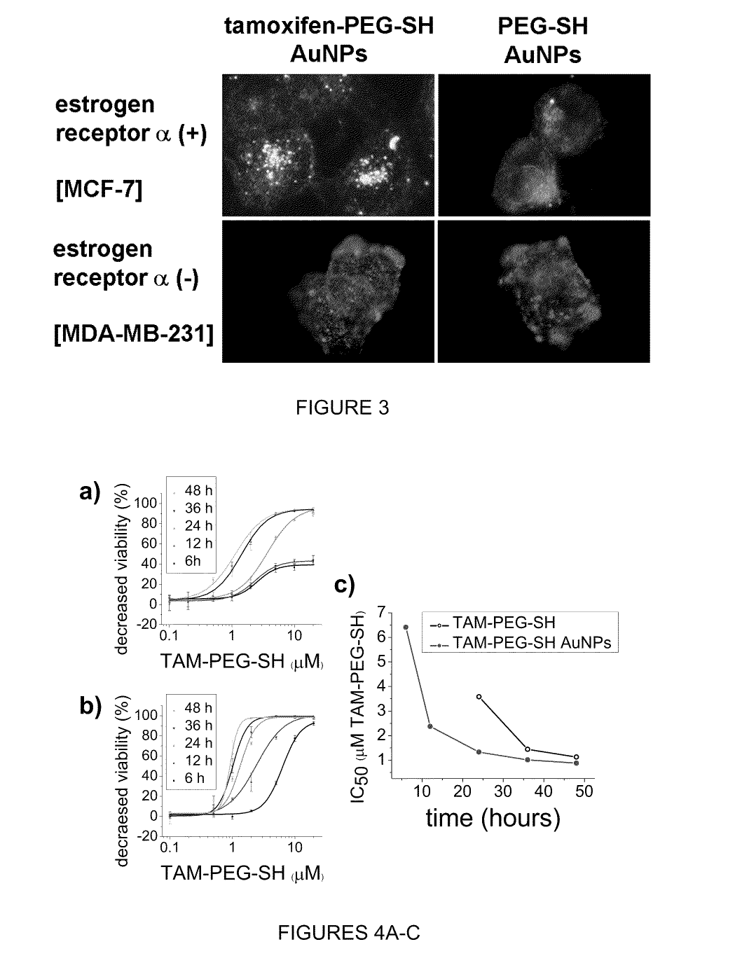

Dark-field scattering microscopy was performed to assess intracellular nanoparticle uptake. FIG. 3 illustrates representative images of ERα(+) [MCF-7, top] and ERα(−) [MDA-MB-231, bottom] breast cancer cells incubated for 24 h with 1 μM TAM-PEG-SH AuNPs and PEG-SH AuNPs. ERα(+) breast cancer cells displayed a high degree of intracellular and perinuclear localization of TAM-PEG-SH AuNPs, while ER(−) breast cancer cells showed no such labeling. These findings are consistent with both reported expression levels and cellular localization (46) of ERα in MCF-7 (47-49) and MDA-MB-231 (48, 50) cell lines. As anticipated (38), AuNPs labeled only with PEG-SH exhibited no apparent cellular labeling or uptake for either ERα(+) or ERα(−) breast cancer cells. Uptake of TAM-PEG-SH AuNPs by ERα(+) breast cells was observed to be time-dependent, with marginal cell surface labeling at 2-6 h and a high degree of perinuclear and cytoplasmic localization at 24 h. To further demonstrate ER expression-dependent targeting, ERα(+) human squamous [HSC-3] oral cancer cells were incubated for 24 h in the presence of 1 μM TAM-PEG-SH AuNPs and PEG-SH AuNPs. Dark-field scattering images from HSC-3 cells show selective uptake of the TAM-PEG-SH AuNPs in a manner similar to that obtained from MCF-7 breast cancer cells. Selected-area optical extinction spectra obtained from the ERα(+) and ERα(−) breast cells exhibited AuNP surface plasmon extinction exclusively from perinuclear regions of ERα(+) cells incubated with TAM-PEG-SH AuNPs (s/n˜10). Extinction from PEG-SH AuNPs was not observed from either cell line.

FIGS. 4A and B illustrate time-dependent dose-response curves for cell viability of ERα(+) MCF-7 breast cancer cells incubated with equivalent concentrations of TAM-PEG-SH as the free drug and the nanoparticle conjugate, respectively. A comparison of the time-dependent IC₅₀ (50% inhibitory concentration) values obtained for the free drug and its AuNP conjugate indicate 1.3-2.7 fold enhanced potency (FIG. 4C) for TAM-PEG-SH AuNPs. While IC₅₀ values for TAM-PEG-SH alone are comparable to or better than those previously reported for MCF-7 breast cancer cells treated with both tamoxifen (43) and its active metabolite (51), a much more dramatic improvement is observed upon nanoparticle ligation, in contrast to the free drug, with significant growth inhibition observed for TAM-PEG-SH AuNPs at both 6 and 12 h incubation (6.4 and 2.4 μM IC₅₀, respectively). In accordance with previous studies (19), no cytotoxic effects were observed in MCF-7 cells treated with PEG-SH AuNPs at the highest concentrations and incubation times used in the present study (P>0.75). Moreover, cytotoxic effects were not observed in ERα(−) MDA-MB-231 breast cancer cells incubated with TAM-PEG-SH alone, PEG-SH AuNPs, or TAM-PEG-SH AuNPs at the highest concentrations and incubation times used in the present study (P>0.28, 0.33, and 0.11, respectively). Although differences in sensitivity to and rates of particle/drug uptake and metabolism for ERα(+) and (−) cell lines may contribute to variation in apparent cytotoxicity, the observed ligand-dependency correlates well with levels of cellular ER expression, particularly under the conditions of extended incubation time and excess concentration used. In addition, cell viability following incubation with TAM-PEG-SH AuNPs in the absence of free drug was found statistically insignificant in difference from that observed in its presence. Here, AuNPs functionalized with TAM-PEG-SH equivalent to that present at the 24 h IC₅₀ of the conjugate in the presence of free drug subsequently exhibited 57±14% cell viability (P>0.6). The lack of significant growth inhibition by the free drug at short incubation times, together with an observed decrease in the disparity between IC₅₀ values of the free drug and the AuNP conjugate over time, and the apparent ligand-dependent response indicate increased rates of TAM-PEG-SH transport by the AuNP conjugate.

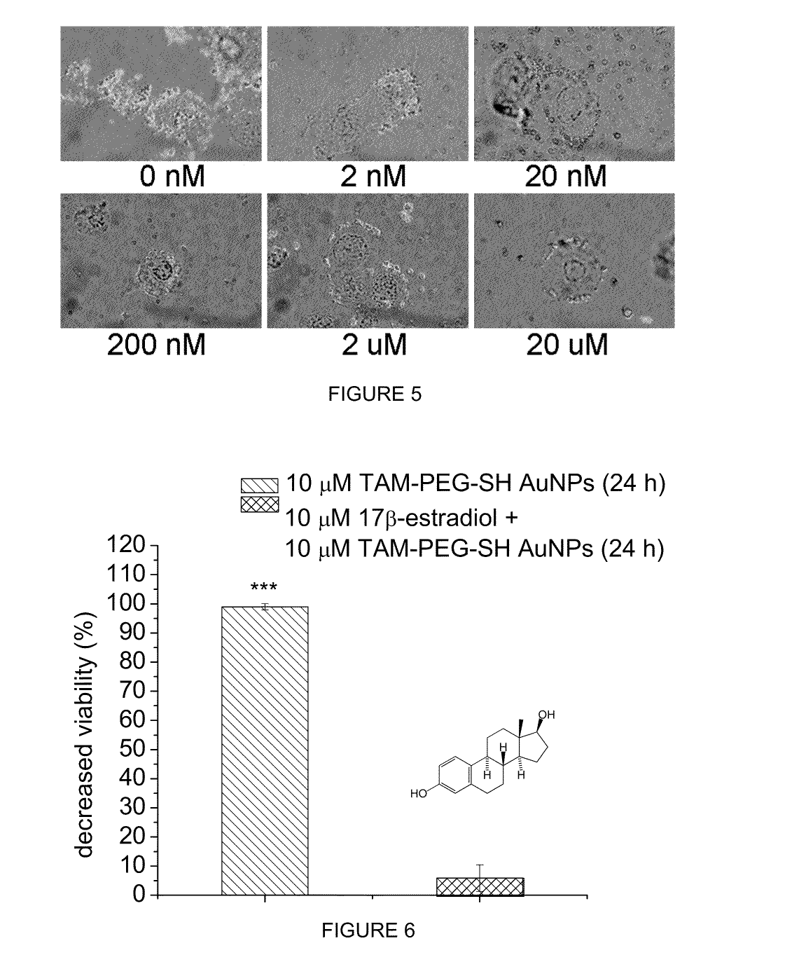

Although our results with the HSC-3 cell line, an ERα(+) oral cancer cell line, further attested to the role of ERα in nanoparticle uptake, it is however conceivable that particle lipophilicity could also contribute to differences cellular uptake and cytotoxicity. In light of this possibility, blocking experiments were performed using ERA's endogenous ligand 17β-estradiol (estrogen) to further confirm receptor-dependent targeting and therapeutic response. ERα(+) MCF-7 breast cells were incubated overnight with increasing concentrations of estrogen, followed by 24 h incubation with 10 μM TAM-PEG-SH AuNPs. Image overlays from bright-field transmission and dark-field scattering microscopy of these cells (FIG. 5) indicate near complete suppression of TAM-PEG-SH AuNP intracellular localization at estrogen concentrations as low as 20 nM. Decreased cell surface labeling was also observed with increasing estrogen concentration. Such competitive effects are in agreement with previous reports indicating 1-2 orders of magnitude greater ERα binding affinity for 17β-estradiol versus TAM (52). Cell viability experiments with ERα(+) breast cells incubated for 24 h with 10 μM TAM-PEG-SH AuNPs and previously blocked overnight with equimolar concentrations of estrogen were also performed (FIG. 6). As in previous studies with the free drug (8), the cytotoxic activity of TAM-labeled AuNPs was near completely suppressed following pre-exposure of the cells to estrogen (P>0.87), while they retained optimal potency in the absence of estrogen (P<0.0001). These findings correlate ERα binding with both TAM-PEG-SH AuNP intracellular localization and subsequent cell death.

The ERα expression-dependent uptake observed here also suggests that the cell membrane-associated receptor may facilitate intracellular nanoparticle transport. Indeed, plasma membrane localized ERα is well documented, as is its recognition of both antibody epitopes for the nuclear receptor and 17β-estradiol in mammalian cells (31, 33). The functions of membrane ERα beyond classical gene transcription, and more recently membrane-initiated signaling, are however less understood (53). Comprehensive studies by Levin and coworkers indicate intracellular transport and caveolar localization of ERα in the plasma membrane of MCF-7 cells in vitro (via caveolin-1 and -2 association) (49). In order to determine whether plasma membrane localized ERα could contribute to receptor-mediated endocytosis of TAM-PEG-SH AuNP conjugates, cytotoxicity was examined under conditions of negligible endocytotic activity. MCF-7 cell viability was shown to increase by 87±2% following incubation with 20 μM TAM-PEG-SH AuNPs for 6 h at 4° C. versus 37° C. (P<0.04), indicating that endocytosis—in addition to ERα binding and intracellular particle delivery—is required for therapeutic response from tamoxifen-labeled AuNP conjugates.

Conclusions. Tamoxifen-gold nanoparticle conjugates were shown to selectively target estrogen receptor alpha in human breast cancer cells with up to 2.7 times enhanced potency in vitro. Optical microscopy and spectroscopy indicate a high degree of perinuclear and cytoplasmic localization of the targeted particles, while neither localization nor cytotoxic effects were observed from the untargeted nanoparticles. Time-dependent dose-response studies show that augmented potency results from increased rates of drug transport by nanoparticle uptake versus passive diffusion of the free drug. Receptor-selective and estrogen-competitive cytotoxicity/uptake of the nanoparticle conjugates indicates no additive effects associated with the gold particles themselves and suggests that plasma membrane-localized ERα may facilitate selective endocytotic transport of these and other therapeutic nanoparticle conjugates. Increased potency and selective intracellular delivery of tamoxifen-gold nanoparticle conjugates provides opportunities for further enhancement by co-functionalization or adjunctive laser photothermal therapy.

Supporting Information Available: Supporting information includes ¹³C and ¹H NMR spectra, TEM and UV-Vis analysis, time-dependent uptake images, oral cancer cell targeting images, selected-area microspectrometry, imaging/spectroscopy of conjugate stability, and estrogen competition images. This material was published in Bioconjugate Chemistry, and the article along with all supporting information is available free of charge via the Internet at http://pubs.acs.org and is expressly incorporated by reference as if fully set forth herein.

### Example 2

### In Vitro Laser Photothermal Therapy of Estrogen Receptor (+) Breast Cancer Cell

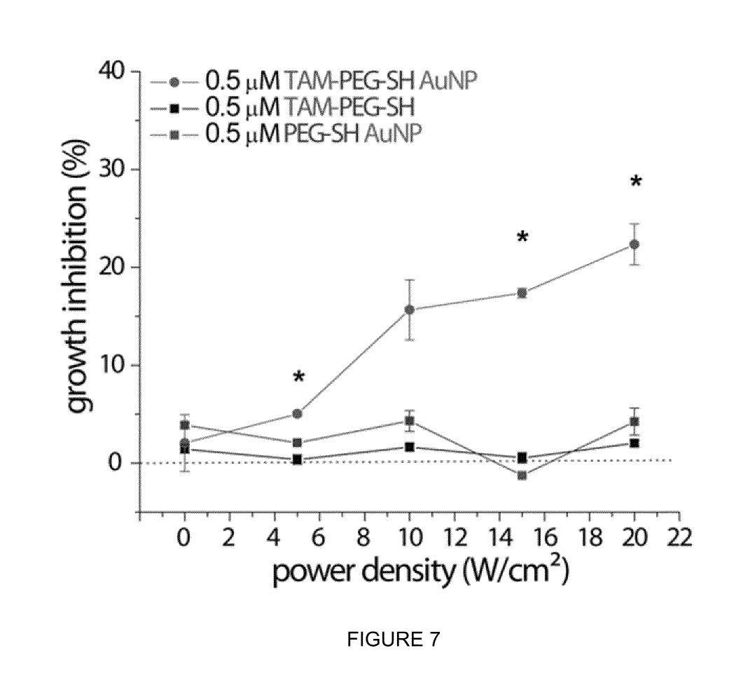

MCF-7 cells were incubated with 0.5 μM TAM-PEG-SH or an equivalent concentration bound to gold nanoparticles (AuNPs) for 24 h to reflect steady-state blood plasma concentrations of tamoxifen administered at 10-20 mg/day (0.40 and 0.81 μM, respectively). Following this incubation, cell cultures ere gently rinsed in sterile DPBS buffer and replaced with unmodified growth media prior to laser photothermal treatment. Laser photothermal treatment was performed using the 514.5 nm line of an argon ion laser (Innova 300 Coherent) for 2 min at room temperature (0.32 cm²). Control cells were similarly removed, however, were untreated with the laser. As shown in FIG. 7, in cells exposed to TAM-PEG-SH bound to gold nanoparticles, a statistically significant increase in growth inhibition was observed commensurate with an increase in the power density of the laser. It is worth noting that in the absence of laser treatment, TAM, TAM-PEG-SH, PEG-SH AuNps and TAM-PEG-SH AuNPs exhibited no appreciable in vitro cytotoxicity at this incubation time and at this concentrations.

### REFERENCES

The below references are hereby incorporated by reference as if fully set forth below.
(1) Obrero, M., Yu, D. V., and Shapiro, D. J. (2002) Estrogen receptor-dependent and estrogen receptor-independent pathways for tamoxifen and 4-hydroxytamoxifen-induced programmed cell death. J. Biol. Chem. 277, 45695-45703.
(2) Bates, S. E., Davidson, N. E., Valverius, E. M., Freter, C. E., Dickson, R. B., Tam, J. P., Kudlow, J. E., Lippman, M. E., and Salomon, D. S. (1988) Expression of Transforming Growth Factor-Alpha and Its Messenger Ribonucleic-Acid in Human-Breast Cancer—Its Regulation by Estrogen and Its Possible Functional-Significance. Mol. Endocrinol. 2, 543-555.
(3) Dubik, D., Dembinski, T. C., and Shiu, R. P. C. (1987) Stimulation of c-Myc Oncogene Expression Associated With Estrogen-Induced Proliferation of Human-Breast Cancer-Cells. Cancer Res. 47, 6517-6521.
(4) Krishnan, V., Wang, X. H., and Safe, S. (1994) Estrogen Receptor-SP1 Complexes Mediate Estrogen-Induced Cathepsin-D Gene-Expression in MCF-7 Human Breast-Cancer Cells. J. Biol. Chem. 269, 15912-15917.
(5) Cleator, S., Heller, W., and Coombes, R. C. (2007) Triple-negative breast cancer: therapeutic options. Lancet Oncol. 8, 235-244.
(6) Jordan, V. C. (2007) Chemoprevention of breast cancer with selective oestrogen-receptor modulators. Nat. Rev. Cancer 7, 46-53.
(7) Shiau, A. K., Barstad, D., Loria, P. M., Cheng, L., Kushner, P. J., Agard, D. A., and Greene, G. L. (1998) The structural basis of estrogen receptor/coactivator recognition and the antagonism of this interaction by tamoxifen. Cell 95, 927-937.
(8) Coezy, E., Borgna, J.-L., and Rochefort, H. (1982) Tamoxifen and Metabolites in MCF₇ Cells: Correlation between Binding to Estrogen Receptor and Inhibition of Cell Growth. Cancer Res. 42, 317-323.
(9) Jordan, V. C. (2003) Tamoxifen: A most unlikely pioneering medicine. Nat. Rev. Drug Discovery 2, 205-213.
(10) Weissleder, R., Kelly, K., Sun, E. Y., Shtatland, T., and Josephson, L. (2005) Cell-specific targeting of nanoparticles by multivalent attachment of small molecules. Nat. Biotechnol. 23, 1418-1423.
(11) Montet, X., Funovics, M., Montet-Abou, K., Weissleder, R., and Josephson, L. (2006) Multivalent Effects of RGD Peptides Obtained by Nanoparticle Display. J. Med. Chem. 49, 6087-6093.
(12) Gestwicki, J. E., Cairo, C. W., Strong, L. E., Oetjen, K. A., and Kiessling, L. L. (2002) Influencing Receptor-Ligand Binding Mechanisms with Multivalent Ligand Architecture. J. Am. Chem. Soc. 124, 14922-14933.
(13) Goodman, C. M., and Rotello, V. M. (2004) Biomacromolecule surface recognition using nanoparticles. Mini-Rev. Org. Chem. 1, 103-114.
(14) Gibson, J. D., Khanal, B. P., and Zubarev, E. R. (2007) Paclitaxel-functionalized gold nanoparticles. J. Am. Chem. Soc. 129, 11653-11661.
(15) Cho, K., Wang, X., Nie, S., Chen, Z., and Shin, D. M. (2008) Therapeutic Nanoparticles for Drug Delivery in Cancer. Clin. Cancer Res. 14, 1310-1316.
(16) Chawla, J. S., and Amiji, M. M. (2002) Biodegradable poly([var epsilon]-caprolactone) nanoparticles for tumor-targeted delivery of tamoxifen. Int. J. Pharm. 249, 127-138.
(17) Maeda, H. (2001) The enhanced permeability and retention (EPR) effect in tumor vasculature: The key role of tumor-selective macromolecular drug targeting. Adv. Enzyme Reg. 41, 189-207.
(18) von Maltzahn, G., Park, J.-H., Agrawal, A., Bandaru, N. K., Das, S. K., Sailor, M. J., and Bhatia, S, N. (2009) Computationally Guided Photothermal Tumor Therapy Using Long-Circulating Gold Nanorod Antennas. Cancer Res. 69, 3892-3900.
(19) Connor, E. E., Mwamuka, J., Gole, A., Murphy, C. J., and Wyatt, M. D. (2005) Gold nanoparticles are taken up by human cells but do not cause acute cytotoxicity. Small 1, 325-327.
(20) Khan, J. A., Pillai, B., Das, T. K., Singh, Y., and Maiti, S. (2007) Molecular effects of uptake of gold nanoparticles in HeLa cells. Chembiochem 8, 1237-1240.
(21) Zhang, F., Skoda, M. W. A., Jacobs, R. M. J., Zorn, S., Martin, R. A., Martin, C. M., Clark, G. F., Goerigk, G., and Schreiber, F. (2007) Gold nanoparticles decorated with oligo(ethylene glycol) thiols: Protein resistance and colloidal stability. J. Phys. Chem. A 111, 12229-12237.
(22) Dickerson, E. B., Dreaden, E. C., Huang, X., El-Sayed, I. H., Chu, H., Pushpanketh, S., McDonald, J. F., and El-Sayed, M. A. (2008) Gold nanorod assisted near-infrared plasmonic photothermal therapy (PPTT) of squamous cell carcinoma in mice. Cancer Lett. 269, 57-66.
(23) O'Neal, D. P., Hirsch, L. R., Halas, N. J., Payne, J. D., and West, J. L. (2004) Photothermal Tumor Ablation in mice using near infrared absorbing nanoshells. Cancer Lett. 209, 171-176
(24) Huang, X., El-Sayed, I. H., and El-Sayed, M. A. (2006) Cancer cell imaging and photothermal therapy in the near-infrared region by using gold nanorods. J. Am. Chem. Soc. 128, 2115-2120.
(25) Huff, T. B., Tong, L., Zhao, Y., Hansen, M. N., Cheng, J. X., and Wei, A. (2007) Hyperthermic effects of gold nanorods on tumor cells. Nanomedicine 2, 125-132.
(26) Hirsch, L. R., Stafford, R. J., Bankson, J. A., Sershen, S. R., Rivera, B., Price, R. E., Hazle, J. D., Halas, N. J., and West, J. L. (2003) Nanoshell-mediated near-infrared thermal therapy of tumors under magnetic resonance guidance. Proc. Natl. Acad. Sci. USA 100, 13549-13554.
(27) El-Sayed, I. H., X., H., and El-Sayed, M. A. (2006) Selective laser photo-thermal therapy of epithelial carcinoma using anti-EGFR antibody conjugated gold nanoparticles. Cancer Lett. 239, 129-135.
(28) Qian, X. M., Peng, X. H., Ansari, D. O., Yin-Goen, Q., Chen, G. Z., Shin, D. M., Yang, L., Young, A. N., Wang, M. D., and Nie, S. M. (2008) In vivo tumor targeting and spectroscopic detection with surface-enhanced Raman nanoparticle tags. Nature Biotechnol. 26, 83-90.
(29) Dixit, V., Van den Bossche, J., Sherman, D. M., Thompson, D. H., and Andres, R. P. (2006) Synthesis and Grafting of Thioctic Acid-PEG-Folate Conjugates onto Au Nanoparticles for Selective Targeting of Folate Receptor-Positive Tumor Cells. Bioconjugate Chem. 17, 603-609.
(30) Tong, L., Zhao, Y., Huff, T. B., Hansen, M. N., Wei, A., and Cheng, J. X. (2007) Gold Nanorods Mediate Tumor Cell Death by Compromising Membrane Integrity. Adv. Mater. 19, 3136-3141.
(31) Levin, E. R. (2005) Integration of the Extranuclear and Nuclear Actions of Estrogen. Mol. Endocrinol. 19, 1951-1959.
(32) Campbell, C. H., Bulayeva, N., Brown, D. B., Gametchu, B., and Watson, C. S. (2002) Regulation of the membrane estrogen receptor-alpha: role of cell density, serum, cell passage number, and estradiol. FASEB J. 16, 1917-1927.
(33) Zivadinovic, D., Gametchu, B., and Watson, C. S. (2005) Membrane estrogen receptor-alpha levels in MCF-7 breast cancer cells predict cAMP and proliferation responses. Breast Cancer Res. 7, R101-R112.
(34) Niidome, T., Yamagata, M., Okamoto, Y., Akiyama, Y., Takahashi, H., Kawano, T., Katayama, Y., and Niidome, Y. (2006) PEG-modified gold nanorods with a stealth character for in vivo applications. J. Controlled Release 114, 343-347.
(35) Jang, S. S., Jang, Y. H., Kim, Y.-H., Goddard, W. A., Flood, A. H., Laursen, B. W., Tseng, H.-R., Stoddart, J. F., Jeppesen, J. O., Choi, J. W., Steuerman, D. W., Delonno, E., and Heath, J. R. (2005) Structures and Properties of Self-Assembled Monolayers of Bistable [2]Rotaxanes on Au (111) Surfaces from Molecular Dynamics Simulations Validated with Experiment. J. Am. Chem. Soc. 127, 1563-1575.
(36) Lavrich, D. J., Wetterer, S. M., Bernasek, S. L., and Scoles, G. (1998) Physisorption and Chemisorption of Alkanethiols and Alkyl Sulfides on Au(111). J. Phys. Chem. B 102, 3456-3465.
(37) Gref, R., Domb, A., Quellec, P., Blunk, T., Muller, R. H., Verbavatz, J. M., and Langer, R. (1995) The Controlled Intravenous Delivery of Drugs Using PEG-Coated Sterically Stabilized Nanospheres. Adv. Drug Delivery Rev. 16, 215-233.
(38) Nativo, P., Prior, I. A., and Brust, M. (2008) Uptake and Intracellular Fate of Surface-Modified Gold Nanoparticles. ACS Nano 2, 1639-1644.
(39) Olofson, R. A., Martz, J. T., Senet, J. P., Piteau, M., and Malfroot, T. (1984) A New Reagent for the Selective, High-Yield N-Dealkylation of Tertiary-Amines—Improved Syntheses of Naltrexone and Nalbuphine. J. Org. Chem. 49, 2081-2082.
(40) Bouzide, A., and Sauve, G. (2002) Silver(I) oxide mediated highly selective monotosylation of symmetrical diols. Application to the synthesis of polysubstituted cyclic ethers. Org. Lett. 4, 2329-2332.
(41) Turkevich, J., Stevenson, P. C., and Hillier, J. (1951) A Study of the Nucleation and Growth Processes in the Synthesis of Colloidal Gold. Discuss. Faraday Soc., 55-75.
(42) Orendorff, C. J., and Murphy, C. J. (2006) Quantitation of Metal Content in the Silver-Assisted Growth of Gold Nanorods. J. Phys. Chem. B 110, 3990-3994.
(43) Guthrie, N., Gapor, A., Chambers, A. F., and Carroll, K. K. (1997) Inhibition of Proliferation of Estrogen Receptor-Negative MDA-MB-435 and -Positive MCF-7 Human Breast Cancer Cells by Palm Oil Tocotrienols and Tamoxifen, Alone and in Combination. J. Nutr. 127, 544S-548S.
(44) Jordan, V. C. (2007) New insights into the metabolism of tamoxifen and its role in the treatment and prevention of breast cancer. Steroids 72, 829-842.
(45) Nozoe, T., Oyama, T., Takenoyama, M., Hanagiri, T., Sugio, K., and Yasumoto, K. (2007) Significance of immunohistochemical expression of estrogen receptors alpha and beta in squamous cell carcinoma of the esophagus. Clin. Cancer Res. 13, 4046-4050.
(46) Htun, H., Holth, L. T., Walker, D., Davie, J. R., and Hager, G. L. (1999) Direct visualization of the human estrogen receptor alpha reveals a role for ligand in the nuclear distribution of the receptor. Mol. Biol. Cell 10, 471-486.
(47) Brooks, S. C., Locke, E. R., and Soule, H. D. (1973) Estrogen Receptor in a Human Cell Line (MCF-7) from Breast Carcinoma. J. Biol. Chem. 248, 6251-6253.
(48) Horwitz, K. B., Zava, D. T., Thilagar, A. K., Jensen, E. M., and McGuire, W. L. (1978) Steroid Receptor Analyses of Nine Human Breast Cancer Cell Lines. Cancer Res. 38, 2434-2437.
(49) Razandi, M., Oh, P., Pedram, A., Schnitzer, J., and Levin, E. R. (2002) ERs associate with and regulate the production of caveolin: Implications for signaling and cellular actions. Mol. Endocrinol. 16, 100-115.
(50) Chen, X. M., Danes, C., Lowe, M., Herliczek, T. W., and Keyomarsi, K. (2000) Activation of the estrogen-signaling pathway by p21(WAF1/CIP1) in estrogen receptor-negative breast cancer cells. J. Natl. Cancer Inst. 92, 1403-1413.
(51) Seeger, H., Diesing, D., Gückel, B., Wallwiener, D., Mueck, A. O., and Huober, J. (2003) Effect of tamoxifen and 2-methoxyestradiol alone and in combination on human breast cancer cell proliferation. J. Steroid Biochem. Mol. Biol. 84, 255-257.
(52) Rich, R. L., Hoth, L. R., Geoghegan, K. F., Brown, T. A., LeMotte, P. K., Simons, S. P., Hensley, P., and Myszka, D. G. (2002) Kinetic analysis of estrogen receptor/ligand interactions. Proc. Natl. Acad. Sci. USA 99, 8562-8567.
(53) Qing, L., Pallas, D. C., Surks, H. K., Baur, W. E., Mendelsohn, M. E., and Karas, R. H. (2004) Striatin assembles a membrane signaling complex necessary for rapid, nongenomic activation of endothelial NO synthase by estrogen receptor alpha. Proc. Natl. Acad. Sci. USA 101, 17126-17131.
(54) Hong, H.-y., Lee, H. Y., Kwak, W., Yoo, J., Na, M.-H., So, I. S., Kwon, T.-H., Park, H.-S., Huh, S., Oh, G. T., Kwon, I.-C., Kim, I.-S., and Lee, B.-H. (2008) Phage display selection of peptides that home to atherosclerotic plaques: IL-4 receptor as a candidate target in atherosclerosis. J. Cell. Mol. Med. 12, 2003-2014.
(55) Park, K., Hong, H.-Y., Moon, H. J., Lee, B.-H., Kim, I.-S., Kwon, I. C., and Rhee, K. (2008) A new atherosclerotic lesion probe based on hydrophobically modified chitosan nanoparticles functionalized by the atherosclerotic plaque targeted peptides. J. Controlled Release 128, 217-223.
(56) Leland, P., Taguchi, J., Husain, S. R., Kreitman, R. J., Pastan, I., and Puri., R. K. (2000) Human breast carcinoma cells express type II IL-4 receptors and are sensitive to antitumor activity of a chimeric IL-4-Pseudomonas exotoxin fusion protein in vitro and in vivo. Mol. Med. 6, 165-178.

What is claimed is:

1. A therapeutic platform for targeted cellular delivery comprising:
a core;
a ligand having specificity for a target; and
a linker comprising a linking moiety,
wherein the linker is attached to the ligand, and wherein the linking moiety attaches the linker to the core, effectively tethering the ligand to the core.

2. The therapeutic platform for targeted cellular delivery of claim 1, wherein the core comprises a gold nanoparticle.

3. The therapeutic platform for targeted cellular delivery of claim 2, wherein the linker comprises a poly(ethylene glycol) derivative having a thiol functional group.

4. The therapeutic platform for targeted cellular delivery of claim 1, wherein the target comprises an endocrine receptor, and wherein the ligand comprises an endocrine receptor antagonist.

5. The therapeutic platform for targeted cellular delivery of claim 4, wherein the endocrine receptor comprises an estrogen receptor, and wherein the ligand comprises tamoxifen.

6. The therapeutic platform for targeted cellular delivery of claim 4, wherein the endocrine receptor comprises an androgen receptor, and wherein the ligand comprises nilutimide.

7. The therapeutic platform for targeted cellular delivery of claim 4, further comprising an active agent.

8. The therapeutic platform for targeted cellular delivery of claim 1, further comprising a targeting moiety.

9. A method for delivering a therapeutic platform to a target cell comprising:
administering to a subject an effective amount of a therapeutic platform, the therapeutic platform comprising:
a core;
a ligand having specificity for a target; and
a linker comprising a linking moiety, wherein the linker is attached to the ligand, and wherein the linking moiety attaches the linker to the core, effectively tethering the ligand to the core; and
selectively targeting the therapeutic platform to a cell of the subject.

10. The method for delivering a therapeutic platform to a target cell of claim 9, wherein the core comprises a gold nanoparticle.

11. The method for delivering a therapeutic platform to a target cell of claim 10, wherein the linker comprises a poly(ethylene glycol) derivative having a thiol functional group.

12. The method for delivering a therapeutic platform to a target cell of claim 11, wherein the subject demonstrates a neoplastic pathology, and wherein the target comprises an endocrine receptor, and wherein the ligand comprises an endocrine receptor antagonist.

13. The method for delivering a therapeutic platform to a target cell of claim 12, wherein the cell comprises a breast cancer cell overexpressing an estrogen receptor, and wherein the endocrine receptor comprises an estrogen receptor, and wherein the ligand comprises tamoxifen.

14. The method for delivering a therapeutic platform to a target cell of claim 13, further comprising inducing selective cytotoxicity of a breast cancer cell overexpressing an estrogen receptor.

15. The method for delivering a therapeutic platform to a target cell of claim 10, further comprising:
exposing the cell to light energy effective to generate heat from the gold nanoparticle; and
thermally ablating the cell.

16. The method for delivering a therapeutic platform to a target cell of claim 15, wherein the cell is selected from the group consisting of a breast cancer cell and a prostate cancer cell.

17. The method for delivering a therapeutic platform to a target cell of claim 9, wherein the therapeutic platform further comprises an active agent.

18. A therapeutic platform for targeted cellular delivery comprising:
a gold nanoparticle;
a ligand having specificity for an endocrine receptor;
a thiol-poly(ethylene glycol) linker, wherein the thiol-poly(ethylene glycol) linker is covalently attached to the gold nanoparticle via a thiol functional group, and wherein the ligand is conjugated to the thiol-poly(ethylene glycol) linker via an azide-alkyne coupling.

19. A therapeutic platform for targeted cellular delivery of claim 18, wherein the ligand comprises tamoxifen, and the endocrine receptor comprises an estrogen receptor.

20. A therapeutic platform for targeted cellular delivery of claim 19, further comprising taxol.
